(19) United States
(12) Patent Application Publication
(10) Pub. No.: US 2007/0078170 A1
(43) Pub. Date: Apr. 5, 2007

(54) Process for the preparation of pioglitazone

(76) Inventors: Chandra Has Khanduri, Haryana (IN); Yatendra Kumar, Haryana (IN); Atulya Kumar Panda, Orissa (IN); Suchitra Chakraborty, Haryana (IN); Mukesh Kumar Sharma, Haryana (IN)

Correspondence Address: RANBAXY INC., 600 COLLEGE ROAD EAST, SUITE 2100, PRINCETON, NJ, 08540, US

(21) Appl. No.: 10/569,999

(22) PCT Filed: Aug. 30, 2004
(86) PCT No.: PCT/IB04/02800

§ 371 Date: Dec. 6, 2006

(30) Foreign Application Priority Data
Aug. 28, 2003 (IN) 1051/DEL/2003

(51) Int. Cl. A61K 31/4439; C07D 417/02

(52) U.S. Cl. 514/342; 546/269.7

(57) ABSTRACT

The present invention relates to a new polymorphic form of 5-[4-[2-(5-ethyl-2-pyridyl)ethoxy]benzyl thiazolidine-2,4-dione of Formula I, commonly known as pioglitazone. It also relates to processes for the preparation of new polymorphic form of pioglitazone and pharmaceutical compositions that include the polymorphic form. It also relates to processes for the preparation of pioglitazone hydrochloride having high solubility and improved bioavailability. It also relates to pharmaceutical compositions that include the pioglitazone hydrochloride having high solubility and a method of treatment of Diabetes type II mellitus comprising administration of the pioglitazone hydrochloride having high solubility.

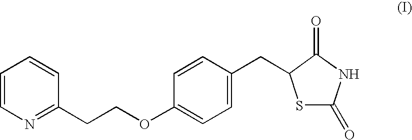

### FIELD OF THE INVENTION

The field of the invention relates to a new polymorphic form of 5-[4-[2-(5-ethyl-2-pyridyl)ethoxy]benzyl thiazolidine-2,4-dione of Formula I, commonly known as pioglitazone. It also relates to processes for the preparation of new polymorphic form of pioglitazone and pharmaceutical compositions that include the polymorphic form. It also relates to processes for the preparation of pioglitazone hydrochloride having high solubility and improved bioavailability. It also relates to pharmaceutical compositions that include the pioglitazone hydrochloride having high solubility and a method of treatment of Diabetes type II mellitus comprising administration of the pioglitazone hydrochloride having high solubility.

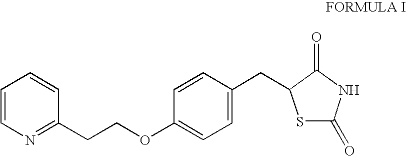

### BACKGROUND OF THE INVENTION

Pioglitazone of Formula I is a thiazolidinedione anti-diabetic agent that uses insulin for its mechanism of action.

Pioglitazone decreases insulin resistance in the periphery and in the liver resulting in increased insulin dependent glucose disposal and decreased hepatic glucose output. Pioglitazone enhances cellular responsiveness to insulin, increases insulin-dependent glucose disposal, improves hepatic sensitivity to insulin and improves dysfunctional glucose homeostasis. In patients with type II diabetes, the decreased insulin resistance produced by pioglitazone results in lower blood glucose concentration, lower plasma insulin levels and lowers HbA values.

U.S. Pat. No. 4,687,777 provides a process for preparation of pioglitazone. Pioglitazone has one chiral centre which gives rise to two stereoisomers. The two isomers of pioglitazone interconvert in vivo after administration.

### SUMMARY OF THE INVENTION

In one general aspect there is provided a new polymorphic form of pioglitazone.

The new polymorphic form of pioglitazone may have, for example, the X-ray powder diffraction pattern of FIG. 1 and infrared spectrum of FIG. 2.

In another general aspect there is provided a pharmaceutical composition that includes a therapeutically acceptable amount of the polymorphic form of pioglitazone; and one or more pharmaceutically acceptable carriers, excipients or diluents.

In another general aspect there is provided a process for the preparation of new polymorphic form of pioglitazone. The process includes obtaining a solution of pioglitazone in one or more non-hydroxylic solvents; and recovering the new polymorphic form of pioglitazone by the removal of the solvent.

The non-hydroxylic solvent may be, for example, one or more of dimethylformamide, chloroform, acetonitrile, tetrahydrofuran, cyclohexane, or mixtures thereof. Removing the solvent may include, for example, one or more of distillation, distillation under vacuum, filtration, filtration under vacuum, decantation and centrifugation.

The process may include further drying of the product obtained.

In one general aspect, the solution of pioglitazone may be obtained by heating the solvent containing pioglitazone. It may be heated from about 40° C. to about 200° C., for example from about 50° C. to about 150° C. It maybe heated from about 10 minutes to about 24 hours.

In another general aspect additional/another solvent may be added to clear solution and it may be cooled before filtration to obtain better yields of the new polymorphic form of pioglitazone.

The second solvent may be one or more of lower alkanol, or mixtures thereof. The lower alkanol may include, for example, one or more of primary, secondary and tertiary alcohol having from one to six carbon atoms. The lower alkanol may include one or more of, for example, methanol, ethanol, denatured spirit, n-propanol, isopropanol, n-butanol, isobutanol, and t-butanol. In particular, the lower alkanol may include, for example, one or more of methanol, ethanol, and denatured spirit.

In another general aspect there is provided a process for preparation of new polymorphic form of pioglitazone. The process includes obtaining a solution of pioglitazone base by treating with an acid; treating the solution with a base; and recovering the new polymorphic form of pioglitazone by the removal of the solvent.

The pioglitazone base may be treated with an acid in presence of an organic solvent to get a clear solution of pioglitazone acid addition salt. Examples of such solvents include lower alkanols such as methanol, ethanol, isopropanol, n-propanol, n-butanol, t-butanol or mixtures thereof. The clear solution can be obtained by heating the mixture of pioglitazone with an acid in presence of an organic solvent.

In one general aspect the solution containing the salt of pioglitazone may be treated with charcoal. The charcoal treatment may be carried out under heating conditions or it may be carried out at a lower temperature.

Examples of acids which can be used in the reaction include inorganic acids such as hydrochloric acid, sulphuric acid, nitric acid, phosphoric acid, or mixtures thereof or organic acids such as formic acid, acetic acid, methane sulphonic acid, 4-toluenesulphonic acid, or mixtures thereof.

Examples of a base which can be used include one or more of primary, secondary or tertiary amines. In particular, triethylamine may be used. Removing the solvent may include one or more of, for example, distillation, distillation under vacuum, filtration, filtration under vacuum, decantation and centrifugation.

The process may include further drying of the product obtained.

In another aspect there is provided pioglitazone hydrochloride having high solubility and improved bioavailability profile.

In another general aspect there is provided a process for the preparation of pioglitazone hydrochloride having high solubility and improved bioavailability profile. The process includes obtaining a solution of pioglitazone base by treating with an acid; isolating pioglitazone hydrochloride from the solution thereof; washing pioglitazone hydrochloride with dilute acid; and drying product to obtain pioglitazone hydrochloride having high solubility and improved bioavailability profile.

Examples of acids which can be for treatment of pioglitazone base include inorganic acids such as hydrochloric acid, sulphuric acid, nitric acid, phosphoric acid, or mixtures thereof.

The details of one or more embodiments of the inventions are set forth in the description below. Other features, objects and advantages of the inventions will be apparent from the description and claims.

### DESCRIPTION OF THE DRAWINGS

FIG. 1 is an X-ray powder diffraction pattern of new polymorphic form of pioglitazone.

FIG. 2 is a Fourier Transform infrared spectrum of new polymorphic form of pioglitazone.

### DETAILED DESCRIPTION OF THE INVENTION

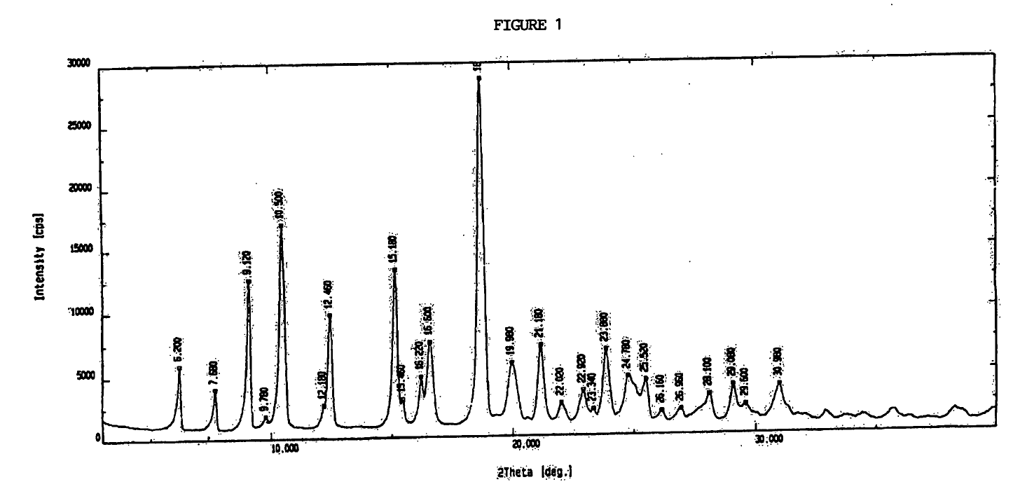

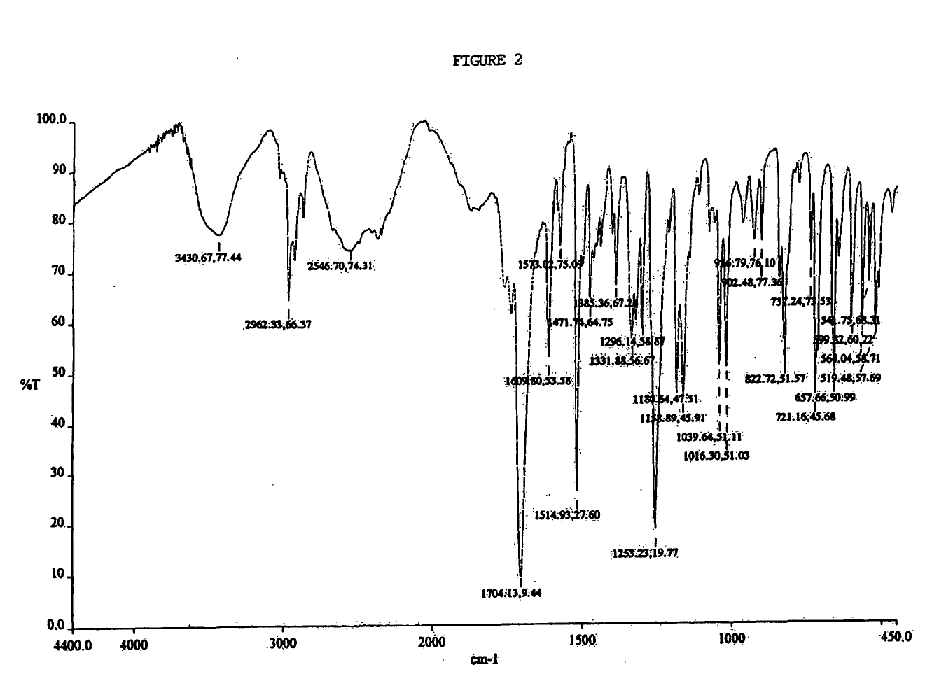

The inventors have found a new polymorphic form of pioglitazone. The new polymorphic form is characterized by its X-ray powder diffraction pattern as shown, for example, in FIG. 1, and infrared spectrum as shown in FIG. 2. The inventors also have developed a process for the preparation of the new polymorphic form of pioglitazone, by preparing a solution of pioglitazone in one or more non-hydroxylic solvents; and recovering the new polymorphic form of pioglitazone by the removal of the solvent. The inventors also have developed pharmaceutical composition that contain the new polymorphic form of the pioglitazone, in admixture with one or more solid or liquid pharmaceutical diluents, carriers, and/or excipients.

In general, the solution of pioglitazone may be obtained by dissolving pioglitazone in a suitable solvent. Alternatively, such a solution may be obtained directly from a reaction mixture in which pioglitazone is formed. The solvent may be removed from the solution by a technique which includes, for example, distillation, distillation under vacuum, filtration, filtration under vacuum, decantation and centrifugation.

Pioglitazone of Formula I may be prepared by subjecting the imino-ether intermediate of Formula II to hydrolysis. The pioglitazone imino-ether of Formula II can be prepared by methods described in U.S. Pat. No. 4,687,777.

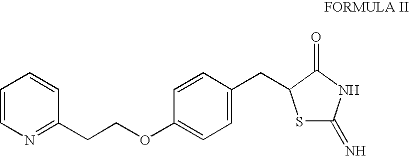

The hydrolysis reaction proceeds advantageously in a proper solvent by employing a mineral acid. Examples of such solvents include alcohols such as methanol, ethanol, propanol, butanol, isobutanol, and 2-methoxyethanol; dimethylsulphoxide, sulfolane, dioxane, tetrahydrofuran, dimethoxyethane; and mixtures thereof. Examples of mineral acids include hydrochloric acid, hydrobromic acid, and sulfuric acid. The reaction may be carried out at a temperature from about 20° C. to about 150° C.

Pioglitazone so obtained may be treated with a non-hydroxylic organic solvent to prepare the new polymorphic form of pioglitazone.

Table 1 gives numerically the spacing “d” in Å, 2θ values and the relative intensity I (%) for exemplary peaks of the X-ray diffraction spectrum of the new polymorphic form of pioglitazone.

| 2θ | d | Intensity | I/I_{O} |
|---|---|---|---|
| 6.20 | 14.2 | 5916 | 21 |
| 7.68 | 11.5 | 3997 | 14 |
| 9.12 | 9.7 | 12632 | 44 |
| 9.78 | 9.0 | 1832 | 6 |
| 10.50 | 8.4 | 17087 | 59 |
| 12.18 | 7.3 | 2652 | 9 |
| 12.46 | 7.1 | 9809 | 34 |
| 15.18 | 5.8 | 13330 | 46 |
| 15.46 | 5.7 | 3075 | 11 |
| 16.22 | 5.5 | 4871 | 17 |
| 16.60 | 5.3 | 7577 | 26 |
| 18.76 | 4.7 | 28759 | 100 |
| 19.98 | 4.4 | 5887 | 20 |
| 21.18 | 4.2 | 7255 | 9 |
| 22.02 | 4.0 | 2610 | 9 |
| 22.92 | 3.9 | 3602 | 13 |
| 23.34 | 3.8 | 2103 | 7 |
| 23.88 | 3.7 | 6842 | 24 |
| 24.78 | 3.6 | 4689 | 16 |
| 25.52 | 3.5 | 4384 | 15 |
| 26.16 | 3.4 | 1857 | 6 |
| 26.96 | 3.3 | 2008 | 7 |
| 28.10 | 3.2 | 3159 | 11 |
| 29.08 | 3.1 | 3889 | 14 |
| 29.60 | 3.0 | 2286 | 8 |
| 30.98 | 2.9 | 3832 | 13 |

Exemplary infrared absorption bands of new polymorphic form of pioglitazone can be observed at (cm⁻¹): 519.48, 541.75, 564.04, 599.82, 657.66, 721.164, 737.24, 822.72, 902.48,926.79, 1016.30, 1039.64, 1158.89, 1180.64, 1296.14, 1331.88, 1385.36, 1471.74, 1573.02, 1609.8, 1519.3, 1704.13, 2546.70, 2962.33, and 3430.67.

The resulting new polymorphic form of pioglitazone may be formulated into ordinary dosage forms such as, for example, tablets, capsules, pills, solutions, etc. In these cases, the medicaments can be prepared by conventional methods with conventional pharmaceutical excipients.

The inventors have also found that the pioglitazone hydrochloride prepared following the process described in U.S. Pat. No. 6,100,403 initially gives a clear solution in acidic media but the solution becomes turbid and the drug precipitates after the solution stands for about 4-5 hours. This affects the bioavailability of the drug considerably.

The inventors have surprisingly found that if pioglitazone hydrochloride prepared by the process of U.S. Pat. No. 6,100,403 is washed with dilute acid instead of ethanol, the pioglitazone hydrochloride so obtained has high solubility, hence the improved bioavailability profile, and the solution remains clear even upon standing for 20 hours. Pioglitazone hydrochloride may be washed with any dilute acid. In particular, it may be washed with 1N HCl.

The resulting pioglitazone hydrochloride may be formulated into ordinary dosage forms such as, for example, tablets, capsules, pills, solutions, etc. In these cases, the medicaments can be prepared by conventional methods with conventional pharmaceutical excipients.

The composition includes dosage forms suitable for oral, buccal, rectal, and parenteral (including subcutaneous, intramuscular, and ophthalmic administration). Dosage forms include solid dosage form, like powder, tablets which can be conventional or sustained release or controlled release, capsules, suppositories, sachets, troches and lozenges as well as liquid suspensions, emulsions, pastes and elixirs. Parenteral dosage forms comprises of intravenous infusions, sterile solutions for intramuscular, subcutaneous or intravenous administration, dry powders to be reconstituted with sterile water for parenteral administration, and the like.

The resulting pioglitazone hydrochloride can be administered for the treatment of diabetes type II mellitus, in a warm-blooded animal.

For the purpose of this disclosure, a warm-blooded animal is a member of the animal kingdom possessed of a homeostatic mechanism and includes mammals and birds.

The present invention is further illustrated by the following examples which are provided merely to be exemplary of the invention and do not limit the scope of the invention. Certain modifications and equivalents will be apparent to those skilled in the art and are intended to be included within the scope of the present invention.

### EXAMPLE 1

Preparation of Pioglitazone

Pioglitazone imino ether of Formula II was added to denatured spirit at room temperature, followed by the addition of concentrated hydrochloric acid. The reaction mixture was heated to reflux for 80-82° C. and stirred at reflux for 18-20 hours. After the completion of the reaction, the reaction mixture was cooled to 40° C. and triethylamine was added at 40-45° C. slowly to adjust pH at 7.3. The reaction mixture was cooled to 50° C. and stirred for 1 hour. The product was isolated by filtration and washed with water and then with denatured spirit at 5-10° C. The product was dried at 45-50° C. till moisture content was not more than 2%.

Yield (%): 51.4-54.3

HPLC quality: 93-95%

### EXAMPLE 2

Preparation of a Novel Polymorphic Form of Pioglitazone

Dimethylformamide (4 lit.) was taken in a round bottom flask and was heated to 85° C. Pioglitazone crude (1 kg) was added under stirring till a clear solution was obtained. The reaction mixture was stirred at 80-85° C. for 10 minutes and cooled to 60° C. Methanol (4 lit.) at 60° C. under stirring was added and the reaction mixture was cooled further to 10° C. The reaction mixture was stirred further at 8-10° C. for 1 hour. The solution was filtered and washed with pre cooled (10° C.) mixture of dimethylformamide: methanol (1:1.2 lit.) by making slurry. The wet solid was transferred to round bottom flask containing preheated dimethylformamide (2.8 lit.) at 85° C. till a clear solution is obtained. The solution was cooled to 60° C. and methanol (2.8 lit.) was charged at 60° C. The reaction mixture was further cooled to 10° C. and was stirred at 5-10° C. for 1 hour. The solution was filtered and washed with pre-cooled (10° C.) mixture of dimethylformamide: methanol (1:1) followed by washing with methanol. The product was air dried at 50-60° C. till loss on drying was not more than 3%.

Yield: 62-67%,

HPLC Purity: 98-99%

### EXAMPLE 3

Preparation of a Novel Polymorphic Form of Pioglitazone

To pioglitazone base (1 Kg), denatured spirit (8 lit) was added, under stirring at room temperature. Concentrated hydrochloric acid (0.322 lit) was added under stirring at 5 25-30° C. and heated to 45° C. till a clear solution was formed. The reaction mixture was stirred for 5 minutes at 40-45° C., followed by the addition of activated charcoal (0.05 kg) at 40-45° C. The reaction mixture was further stirred for 30 minutes at the same temperature, filtered through celite bed at 40-45° C. and the bed was washed with denatured spirit (1 lit). Triethylamine (0.57 lit) was added lot wise at 40-45° C. to adjust pH around 7.3. The solution was cooled to 10° C., stirred at 5-10° C. for 1 hour, and filtered. The solid was washed with pre cooled (5-10° C.) denatured spirit (2 lit) by making a slurry. The product was dried under vacuum at 55-60° C. till moisture content was not more than 3%.

Yield: 86.6-91.3%,

HPLC Purity: 98.5-99.0%

### EXAMPLE 4

Conversion of Pioglitazone to Pioglitazone Hydrochloride

To pioglitazone (1 Kg), de-ionized water (7 lit.) was added under stirring at 80° C., followed by stirring at 80-82° C. for 10 minutes. To the above reaction mixture, concentrated hydrochloric acid (0.41 lit.) was added under stirring at 80° C., followed by stirring at 80-82° C. for 10 minutes. Activated charcoal was added to the mixture at 80-82° C., and the mixture was stirred for 30 minutes at 80-82° C. The solution was filtered through celite bed directly and was washed with water (1 lit.) at 80-82° C. The solution was cooled to 10° C. and was stirred at 5-10° C. for 1 hour, and filtered.

The filtered solid was added to ethanol in Round bottom flask under stirring at room temperature and heating to 80° C. The mixture was stirred for 15 minutes and was cooled to 10° C., followed by stirring at 5-10° C. for 30 minutes. The solid was filtered under nitrogen atmosphere and was washed with ethanol (0.6 lit.) by making slurry at 5-10° C. The product was dried under vacuum till moisture content was less than 0.5%

Yield (%): 68.3%

HPLC Purity: 99.5%

### EXAMPLE 5

Conversion of Pioglitazone to Pioglitazone Hydrochloride

Pioglitazone hydrochloride was prepared in a similar manner as exemplified in Example 4. To de-ionized water (7 L) heated to 80° C. added the product obtained from Example 4 (1 Kg) under stirring at 80° C. It was further stirred for 10 min at 80 - 82° C. and conc. HCI (0.5 L) was added to it under stirring at 80° C. The resultant mass was further stirred for 10 min at 80-82° C. to obtain a clear solution. The solution was filtered through celite bed directly into RB flask and the bed was washed with IN HCI (1 L) at 80-82° C. It was cooled to 5° C. under stirring at 5-0° C. for 1 hr. The solids were filtered under N₂ atmosphere and dried on the Büchner funnel for about 15 minutes. The product obtained was then dried under vacuum at 55-60° C. to afford the title compound.

Yield: 930.0 g (93%)

HPLC Purity: 99.61%

Samples of pioglitazone hydrochloride prepared in examples 4 and 5 were studied for their solubility, as set forth below:

### EXAMPLE 6

Solubility Studies

Solubility experiments were performed in USPII dissolution apparatus operated at 150 r.p.m., at 25° C., in the following media: 250 ml of 0.01N HC1-0.3M KCI (pH 2)

Procedure: 625 mg of pioglitazone hydrochloride was added in 250 ml of media and solubility was determined at given time points.

Pioglitazone Hydrochloride (Solubility (in mg/ml)
(Average of six unit data)

| Time | | Example 4 | Example 5 |
|---|---|---|---|
| 30 | min | — | 2.719 |
| 1.5 | hrs | 2.265 | 2.587 |
| 5 | hrs | 0.722 | 2.766 |
| 20 | hrs | 0.473 | 2.747 |

Observation: For pioglitazone hydrochloride of Example 4, the media starts becoming turbid and shows drug precipitation on later time points.

These data indicate that the pioglitazone hydrochloride prepared as, for example, in Example 5 offers increased solubility which allows it to be formulated in various desirable routes, such as a low volume injectable, rapid oral dissolve or a controlled- or extended release dosage form. The pioglitazone hydrochloride described herein was found to have a solubility of more than 2.4 mg/ml in 0.01N HC1-0.3M KC1 media (pH 2) media and it does not precipitate from the solution upon standing for more than 5 hours.

While the present invention has been described in terms of its specific embodiments, certain modifications and equivalents will be apparent to those skilled in the art and are included within the scope of the present invention.

We claim:

1. A polymorphic form of pioglitazone characterized by X-ray diffraction pattern having peaks at about 9.1, 10.5, 12.5, 15.2, 16.2, 16.6, 18.8, 20.0 and 21.2 degrees 2θ.

2. The polymorphic form of pioglitazone of claim 1, wherein the pioglitazone has infrared absorption bands at about 519, 564, 658, 721, 823, 1016, 1040, 1159, 1181, 1253, 1704, 2547, 2962, and 3431 cm⁻¹.

3. A pharmaceutical comprising:
a therapeutically effective amount of polymorphic form of pioglitazone having an X-ray diffraction pattern having peaks at about 9.1, 10.5, 12.5, 15.2, 16.2, 16.6, 18.8, 20.0 and 21.2 degrees 2θ,
and one or more pharmaceutically acceptable carriers, excipients or diluents.

4. The pharmaceutical composition of claim 3, wherein the pioglitazone has infrared absorption bands at about 519, 564, 658, 721, 823, 1016, 1040, 1159, 1181, 1253, 1704, 2547, 2962, and 3431 cm⁻¹.

5. A process for the preparation of a polymorphic form of pioglitazone, the process comprising:
obtaining a solution of pioglitazone in one or more non-hydroxylic solvents; and
recovering the polymorphic form of pioglitazone by the removal of the solvent.

6. The process of claim 5, wherein the non-hydroxylic solvent comprises one or more of dimethylformamide, chloroform, acetonitrile, tetrahydrofuran, cyclohexane or mixtures thereof.

7. (canceled)

8. (canceled)

9. (canceled)

10. (canceled)

11. The process of claim 5, wherein removing the solvent comprises one or more of distillation, distillation under vacuum, filtration, filtration under vacuum, decantation and centrifugation.

12. The process of claim 5 further comprising adding second solvent before removing the solvent.

13. The process of claim 12, wherein the second solvent comprises one or more of methanol, ethanol, isopropanol, n-propanol, n-butanol, t-butanol or mixtures thereof.

14. (canceled)

15. A process for the preparation of a polymorphic form of pioglitazone, the process comprising:
obtaining a solution of pioglitazone base by treating with an acid; treating the solution with a base; and recovering the polymorphic form of pioglitazone by the removal of the solvent.

16. The process of claim 15, wherein the acid comprises one or more of inorganic acid or organic acid.

17. The process of claim 16, wherein the inorganic acid comprises one or more of hydrochloric acid, sulphuric acid, nitric acid, phosphoric acid or mixtures thereof.

18. The process of claim 17, wherein the inorganic acid is hydrochloric acid.

19. The process of claim 16, wherein the organic acid comprises one or more of formic acid, acetic acid, methane sulphonic acid, 4-toluenesulphonic acid or mixtures thereof.

20. The process of claim 15 wherein the solution of pioglitazone base is obtained in a solvent.

21. The process of claim 20, wherein the solvent comprises one or more of lower alkanols.

22. The process of claim 21, wherein the lower alkanol comprises one or more of methanol, ethanol, denatured spirit, isopropanol, n-propanol, n-butanol, or t-butanol.

23. (canceled)

24. The process of claim 15, wherein the base comprises one or more of primary, secondary or tertiary amine.

25. The process of claim 24, wherein the base is triethylamine.

26. The process of claim 15, wherein removing the solvent comprises one or more of distillation, distillation under vacuum, filtration, filtration under vacuum, decantation and centrifugation.

27. Pioglitazone hydrochloride having a solubility of more than about 2.4 mg/ml in 0.01N HC1-0.3M KCl media (pH 2) media.

28. The pioglitazone hydrochloride of claim 27, wherein the solubility is from about 2.4 mg/ml to about 2.8 mg/ml.

29. The pioglitazone hydrochloride of claim 27, wherein a solution of pioglitazone hydrochloride in 0.01N HC1-0.3M KCl media (pH 2) media does not precipitate upon standing for more than 5 hours.

30. A process for the preparation of pioglitazone hydrochloride having a solubility of more than about 2.4 mg/ml in 0.01N HC1-0.3M KCl media (pH 2) media, the process comprising:
obtaining a solution of pioglitazone base by treating with an acid; isolating pioglitazone hydrochloride from the solution thereof; washing pioglitazone hydrochloride with dilute acid; and drying product to obtain pioglitazone hydrochloride having high solubility.

31. The process of claim 30, wherein the acid comprises one or more of hydrochloric acid, sulphuric acid, nitric acid, phosphoric acid or mixtures thereof.

32. The process of claim 31, wherein the acid is hydrochloric acid.

33. The process of claim 30, wherein the pioglitazone hydrochloride is washed with 1N HCl.

34. A pharmaceutical comprising:
a therapeutically effective amount of pioglitazone hydrochloride having a solubility of more than about 2.4 mg/ml in 0.01N HC1-0.3M KC1 media (pH 2) media;
and one or more pharmaceutically acceptable carriers, excipients or diluents.

35. A method of treating type II diabetes mellitus in a warm-blooded animal comprising administering a pharmaceutical composition that includes pioglitazone hydrochloride having a solubility of more than about 2.4 mg/ml in 0.01N HC1-0.3M KCl media (pH 2) media.
